(19) United States
(12) Patent Application Publication
(10) Pub. No.: US 2021/0093810 A1
(43) Pub. Date: Apr. 1, 2021

(54) MINIATURIZED INHALATION DEVICE

(71) Applicant: Aspuraclip GmbH, Schönefeld (DE)

(72) Inventors: Wolfgang Kleiner, Schönefeld (DE); Vinh-Nghi Tiet, Schönefeld (DE); Manfred Wollmershäuser, Schönefeld (DE)

(21) Appl. No.: 17/032,108

(22) Filed: Sep. 25, 2020

(30) Foreign Application Priority Data
Sep. 26, 2019 (DE) 10 2019 126 003.5

(51) Int. Cl. A61M 15/08; A61M 15/00

(52) U.S. Cl. CPC A61M 15/085; A61M 15/0003

(57) ABSTRACT

Provided is a miniaturized inhalation device for inhaling at least one active ingredient through the nose, including: a U-shaped component and a first and second elongate reservoir portion which each have a cavity for receiving an active ingredient and being configured to be introduced into a nostril. The cavities each have an opening on the top side through which the active ingredient can exit from the corresponding cavity. The inhalation device also has a curved bracket portion which integrally connects the first and the second elongate reservoir portion and extends at least in a base region of the U-shaped component, and a self-holding mechanism to be held against the nose, and an integral clamping projection is arranged on the inside in the region of the first and the second elongate reservoir portion. A set comprising at least one inhalation device and one storage container may also be provided.

### CROSS-REFERENCE TO RELATED APPLICATIONS

This application claims priority to German Application No. 10 2019 126 003.5, having a filing date of Sep. 26, 2019, the entire contents of which are hereby incorporated by reference.

### FIELD OF TECHNOLOGY

The following relates to a miniaturized inhalation device for inhaling at least one active ingredient through the nose.

### BACKGROUND

A miniaturized inhalation device for inhaling an active ingredient through the nose is known from document DE 20 2011 102 694 U1. The inhalation device in miniature format consists of a housing and an active ingredient reservoir. The housing, the larger part of which is intended to be placed inside the nose, is made of an elastic U-shaped tube in which there are cavities that are filled with an active ingredient reservoir made of a fibrous or porous material.

Miniaturized inhalation devices are also described in documents DE 10 2014 014 684 A1 and DE 20 2013 000 744 U1.

### SUMMARY

An aspect relates to a miniaturized inhalation device for inhaling at least one active ingredient through the nose (nasal inhalation or nasal respiration), which is configured in an improved manner for inhaling the active ingredient through the nose, in particular with regard to the provision of the active ingredient to be inhaled and the ability of the device to stay on the nose.

According to one aspect, a miniaturized inhalation device for inhaling at least one active ingredient through the nose is provided, which has a substantially U-shaped component and is formed comprising the following features: a first elongate reservoir portion which has a first cavity for receiving a first amount of an active ingredient and is configured is to be introduced into a first nostril, wherein the first cavity has a first top side opening through which the active ingredient can exit from the first cavity; a second elongate reservoir portion which has a second cavity, which is separate from the first cavity, for receiving a second amount of the active ingredient or a different active ingredient and is configured to be introduced into a second nostril, wherein the second cavity has a second top side opening through which the active ingredient and/or the different active ingredient can exit from the second cavity; a curved bracket portion which integrally connects the first and the second elongate reservoir portion and extends at least in a base region of the U-shaped component; and a self-holding mechanism to be held against the nose, in which mechanism the first and the second elongate reservoir portion are elastically pretensioned by the curved bracket portion against a bending up movement which increases the distance between the first and the second elongate reservoir portion and an integral clamping projection is arranged on the inside in the region of the first and the second elongate reservoir portion, which projections engage opposite sides of the nasal septum for holding.

The active ingredient is provided in the cavities in liquid form (liquid quantity or sample) and can thus be inhaled through each cavity opening.

The first and second elongate reservoir portions and the curved bracket portion may form an integral body together with the U-shaped component, which consists of a plastics material. A resin material or an elastomer may, for example, be used as the plastics material. Plastics materials are used in this case, for example, that are approved for use in connection with food.

The curved bracket portion may be flat in the cross section. The flat material can be free of cavities. The flat material does not, in particular, have any cavities which form a fluid connection between the first and the second cavity. In the various embodiments, these are always separated from one another without there being a fluid connection between the first and the second cavity in the first/second elongate reservoir portion.

The curved bracket portion may be molded onto the inside of the first and the second elongate reservoir portions, respectively, for example via a portion below the first/second cavity that tapers towards the inside. This portion tapering towards the inside is integrally connected to both the elongate reservoir portion and to an associated end of the curved bracket portion. In alternative embodiments, the portion between the elongate reservoir portion and the curved bracket portion may taper towards the center or towards the outside such that the bracket portion is integrally molded to each elongate reservoir portion in the center or the outside. The tapering from the elongate reservoir portion to the curved bracket portion that is configured to be a flat shape or a different shape which has a reduced component thickness compared to the elongate reservoir portions is characteristic. In these or other embodiments, the integral body of the miniaturized inhalation device may be formed from the plastics material as an injection-molded component.

The curved bracket portion may be integrally molded on the inside of the first and the second elongate reservoir portions, respectively.

The first and the second elongate reservoir portions may each have a cylindrical shape, for example a round, cylindrical shape.

The first and the second cavities may each have a cylindrical shape. The first and the second cavity can be configured as cavities having a round, cylindrical shape.

A projection tip of each clamping projection may be rounded. As an alternative or in addition, it can be provided that the transitions between the clamping projection and the elongate reservoir portion arranged on both sides of the projection tip of the clamping projection are each rounded, at least when the U-shaped component is viewed from the front.

Each clamping projection may have a triangular shape when the U-shaped component is viewed from the front. Alternatively, a semicircular shape or another polygonal shape may be provided, each having rounded corners.

Each clamping projection may be formed or arranged such that it completely overlaps with the first/second elongate reservoir portion in the longitudinal direction. In this case, each clamping projection and the first/second elongate reservoir portion may have substantially the same length in the longitudinal direction. It can also be provided that the clamping projection is shorter in the longitudinal direction than the first/second elongate reservoir portion.

When the U-shaped component is viewed from above, each clamping projection may have a smaller component thickness than the first/second elongate reservoir portion. Alternatively, each clamping projection may be configured having a component thickness which is substantially the same as the associated elongate reservoir portion. In this or other embodiments, each clamping projection may be configured having a substantially completely rounded surface.

Each clamping projection may be arranged below and at a distance from a circumferential edge of the first/second top side opening. Alternatively, an extension of the clamping projection on the base side may extend as far as the circumferential edge of the associated top side opening, for example tapering completely at the edge.

It may be provided that the first cavity is provided with a first closure made of material permeable to the active ingredient and the second cavity is provided with a second closure made of material permeable to the active ingredient and/or the different active ingredient. In order to ensure the release of the active ingredient for inhalation in the nostrils, the material of the first and the second closure, which may be the same material or different materials, is permeable to the active ingredient or active ingredients, in particular in vaporized or volatile form. It may be a porous or fiber-like material, for example. The cavities closed by each closure (outside of the closure itself) are free from an insert arranged therein, for example an insert made from material which contains the active ingredient in absorbed form (active ingredient reservoir).

The extension of the first and the second closure of the first/second elongate reservoir portion in the longitudinal direction may be limited to a region above each clamping projection. In this case, each clamping projection and the closure arranged in the associated elongate reservoir portion do not substantially overlap in the longitudinal direction of the elongate reservoir portion. This means that the closure is arranged completely above the clamping projection.

The first and the second closure may be arranged such that they are set back with respect to the first and the second top side opening. Each closure may be partially or completely set back by the thickness of the edge thickening. Alternatively, it may be provided that the closure is set back with respect to the top side opening by a distance which is greater than the thickness of the edge thickening.

A plug, in which the active ingredient and/or the different active ingredient is placed such that it can be dispensed, may be arranged in the first and/or the second cavity. In that case, the plug, which is releasably introduced into the associated cavity, may, for example, completely or partially fill the cavity. The plug may be wholly or partially made of an absorbent material which contains and soaks up each active ingredient in such a way that it can then escape through the opening of the cavity during use. It may be a porous or fiber-like material, for example.

The self-holding mechanism may have an outer edge thickening along the circumferential edge of the first and the second top side opening. The outer edge thickening may be formed around the top side opening such that it is continuous or discontinuous. The edge thickening may have a rounded outer surface.

The first and the second top side opening may have an opening cross section which is substantially the same as a cavity cross section in the region of the first and the second cavity. Alternatively, the opening cross section of the top side opening may be larger than the cavity cross section. In this embodiment, the closure may be arranged in a portion having the larger opening cross section. At the transition between the different opening cross sections, a step may be provided as a stop for the closure.

According to one aspect, a storage container may be provided for the inhalation device. Accordingly, a set may be provided which has at least one miniaturized inhalation device as described above and a storage container. The storage container is configured to accommodate the inhalation device, the storage container comprising a first receptacle and a second receptacle which are dimensioned and shaped to at least partly accommodate the first elongate reservoir portion and the second elongate reservoir portion such that the first and second cavity are sealed off from the environment.

Sealed means or a seal or a sealant in particular that the active ingredient or ingredients do not arrive in the environment in a gaseous or volatile form as soon as the inhalation device is inserted into the storage container and stored therein when not in use. In this way, the shelf life can be extended since the seal is able to prevent or at least reduce the volatilization of the active ingredient when not in use. The storage container can therefore also be referred to as a “fragrance case.” Before using the inhalation device for the first time, it is accordingly advantageous to provide the device in a sealed manner. A set having a plurality of (same or different) inhalation devices and a storage container may be provided.

To obtain a seal, it can simply be provided that each of the openings on the top side are pressed against an end-face wall of each receptacle and thus sealed, for example when a lid of the container is closed and the inhalation device is thus pressed into an end position or at least held in this position. Furthermore (or also alternatively), the receptacles are configured in such a way that a corresponding seal is formed by the circumferential thickening described above. In other words, an inner diameter of the receptacles, in particular in an end region in which each edge thickening is located when the inhaler is stored in the storage container, is equal to or somewhat smaller than an outer diameter of the edge thickening in order to press the edge thickening against the inner wall of the receptacles and thus achieve an improved seal.

The storage container advantageously has a lid, for example a hinged lid, which may snap into place to close. Any other lid designs are conceivable as well, however. Closing the lid prevents the inhaler from falling out. However, it may also be provided that the storage container can be closed by the lid in such a way that a further seal against the environment is formed. In addition, the storage container may have additional features, such as an eyelet to attach the container to a key ring, a chain or the like.

### BRIEF DESCRIPTION

Some of the embodiments will be described in detail, with reference to the following figures, wherein like designations denote like members, wherein:

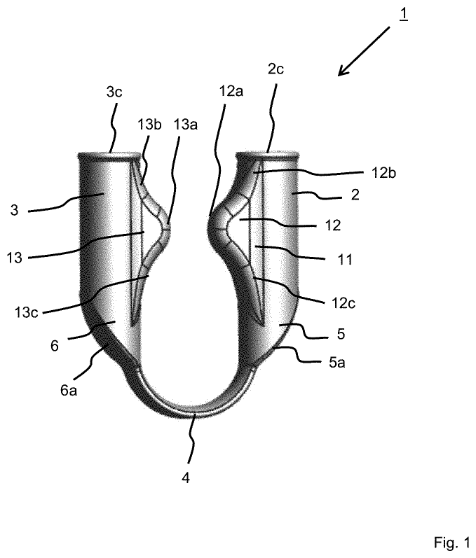

FIG. 1 shows a schematic perspective view of a miniaturized inhalation device from the front;

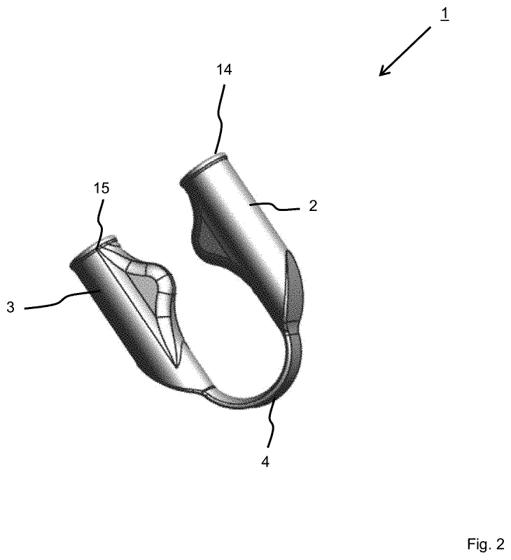

FIG. 2 shows a schematic perspective view of the miniaturized inhalation device of FIG. 1 obliquely from the front;

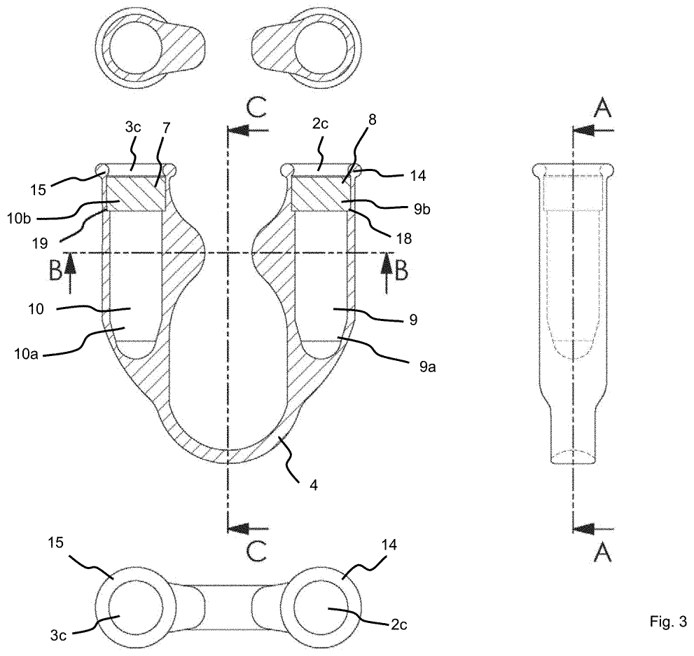

FIG. 3 shows a schematic view of an embodiment of a miniaturized inhalation device in cross section;

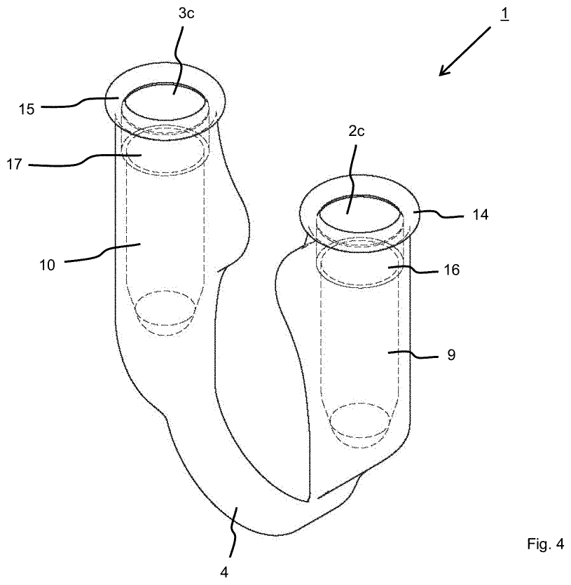

FIG. 4 shows a schematic perspective view of the miniaturized inhalation device of FIG. 3;

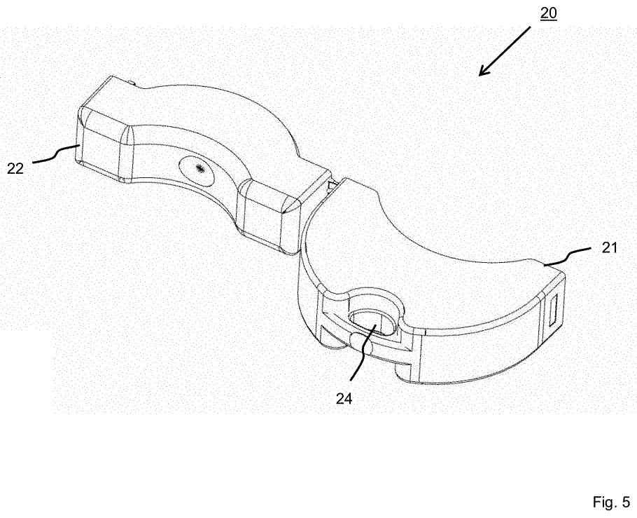

FIG. 5 shows a schematic perspective view of a storage container obliquely from above;

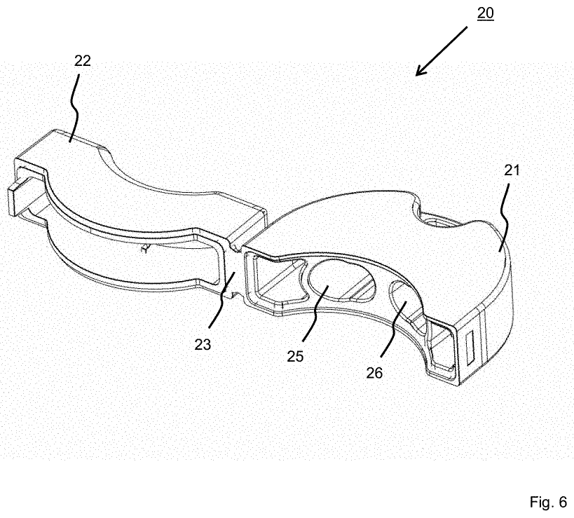

FIG. 6 shows a schematic perspective view of the storage container of FIG. 5 obliquely from below; and

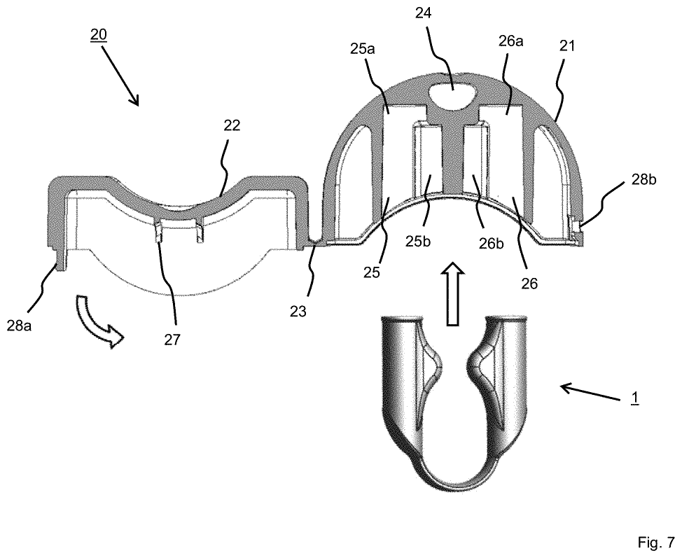

FIG. 7 shows a schematic view of the storage container of FIG. 5 in cross section.

### DETAILED DESCRIPTION

FIGS. 1 and 2 show perspective views of a miniaturized inhalation device 1 comprising a first and a second elongate reservoir portion 2, 3 which are integrally interconnected by a curved bracket portion 4. The curved bracket portion 4 has a flat cross section in the embodiment shown. The curved bracket portion 4 extends at least in a lower region of the U-shaped component of the miniaturized inhalation device 1.

The curved bracket portion 4 is integrally molded on the inside of the first and the second elongate reservoir portion 2, 3. It is molded on each portion 5, 6 which tapers towards the curved bracket portion 4. In the embodiment shown, the tapering portion 5, 6 forms a lower portion 2a, 3a of the elongate reservoir portions 2, 3 and is flattened on an outer side 5a, 6a.

The elongate reservoir portions 2, 3 have a round, cylindrical shape. Each opening 2c, 3c on the top side is provided at the upper end 2b, 3b, through which an active ingredient for inhalation can exit when the miniaturized inhalation device 1 together with the elongate reservoir portions 2, 3 are at least partially inserted into a nose (not shown).

The active ingredient to be inhaled is received in cavities 9, 10 which are arranged in the elongate reservoir portions 2, 3, for example in the form of cylindrical cavities, which are in fluid communication with the openings 2c, 3c. A plug, which completely or partially fills the cavity 9, 10, may be arranged in each cavity 9, 10 (not shown). The plug may be wholly or partially made of an absorbent material which contains and soaks up each active ingredient in such a way that it can then escape through the opening 2c, 3c of the cavity 9, 10 during use.

In order to hold the miniaturized inhalation device 1 on a nose, a clamping mechanism 11 having clamping projections 12, 13 is provided, which are arranged on the inside of the elongate reservoir portions 2, 3 and opposite one another. In the embodiment shown, the clamping projections 12, 13 comprise, when viewed from the front of the miniaturized inhalation device 1, a substantially triangular design, a projection tip 12a, 13a that is rounded, which is also the case for transitions 12b, 12c, 13b, 13c between the clamping projections 12, 13 and the elongate reservoir portions 2, 3.

The clamping projections 12, 13 are arranged in the region of the cavities 9, 10 and below an edge thickening 14, 15 which surrounds each opening 2c, 3c on the top side.

FIGS. 3 and 4 show a further embodiment of a miniaturized inhalation device in section and in perspective. The same reference numerals are used for the same features as in connection with FIGS. 1 and 2. The openings 2c, 3c on the top side are surrounded by the sealing edge thickening 14, 15. The cavities 9, 10 taper in a lower portion 9a, 10a. In an upper portion 9b, 10b, an expansion region 16, 17 is formed which serves to accommodate a closure 7, 8 in each case. The closures 7, 8 are permeable for inhalation of each active ingredient to be dispensed. The active ingredient to be inhaled can then be received in the cavities 9, 10 in liquid form or, as described above, absorbed in a plug. The outer circumferential wall is thinned in the extension region 16, 17 in each case.

At the transition between the lower portion 9a, 10a and the relevant upper portion 9b, 10b, or at the lower end of each expansion region 16, 17, a step 18, 19 is formed in the embodiment shown, which defines a stop for each closure 7, 8. This prevents the closures 7, 8 from entering too deeply into each cavity 9, 10, in particular into the lower portion 9a, 10a, which is provided for the liquid active ingredient or is filled with the ingredient.

FIGS. 5 and 6 show perspective views of a storage container 20. In FIG. 7, it is shown in cross section and together with an inhalation device 1. The inhalation device 1 may be stored in the container 20, in particular when not in use and after a sealed packaging has been opened for the first time, in order to protect the inhalation device 1 and improve its shelf life, for example to preserve a fragrance of the active ingredient (hence also called “fragrance case”). For this purpose, the inhalation device 1 is inserted into an accommodating part 21 of the container 20 and the lid 22 is closed (cf. the corresponding arrows in FIG. 7). In this embodiment, the lid 22 is connected to the accommodating part 21 by a film hinge 23 and has a latching lug 28a which can latch into a corresponding recess 28b in the housing part in order to close. An eyelet 24 may be used, for example, to attach the device to a key ring.

The accommodating part 21 has two elongate receptacles 25, 26 which are adapted to the two elongate reservoir portions 2, 3 of the inhalation device 1 to be accommodated. In particular, each edge thickening 14, 15 comes into contact with a circumferential inner wall in an end region 25a, 26a of the receptacles 25, 26 such that the cavities 9, 10 (for all of the above-described embodiments of the inhalation device) are sealed against the environment and the active ingredient is preserved longer than when stored openly. The edge thickenings 14, 15 may also be pressed against the respective end of the receptacles 25, 26 for a further sealing effect when the container 20 is closed. For this purpose, corresponding structures 27 may be provided in the lid 22 which, in the closed state, press against the bracket portion 4 of the inhalation device 1. The receptacles 25, 26 also have corresponding accommodating regions 25b, 26b for the clamping projections 12, 13.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality.

1. A miniaturized inhalation device for inhaling at least one active ingredient through the nose, comprising a substantially U-shaped component and
a first elongate reservoir portion which has a first cavity for receiving a first amount of an active ingredient and is configured to be introduced into a first nostril, wherein the first cavity has a first top side opening through which the active ingredient can exit from the first cavity;
a second elongate reservoir portion (3) which has a second cavity, which is separate from the first cavity, for receiving a second amount of the active ingredient or a different active ingredient and is configured to be introduced into a second nostril, wherein the second cavity has a second top side opening through which the active ingredient and/or the different active ingredient can exit from the second cavity;
a curved bracket portion which integrally connects the first and the second elongate reservoir portion and extends at least in a base region of the U-shaped component; and
a self-holding mechanism to be held against the nose, in which mechanism the first and the second elongate reservoir portion are elastically pretensioned by the curved bracket portion against a bending up movement which increases the distance between the first and the second elongate reservoir portion and an integral clamping projection is respectively arranged on the inside in the region of the first and the second elongate reservoir portion, which projections engage opposite sides of the nasal septum for holding.

2. The miniaturized inhalation device according to claim 1, wherein the first and the second elongate reservoir portions and the curved bracket portion form an integral body together with the U-shaped component, which consists of a plastics material.

3. The miniaturized inhalation device according to claim 1, wherein the curved bracket portion is flat in the cross section.

4. Miniaturized inhalation device according to claim 1, wherein the curved bracket portion is integrally molded on the inside of the first and the second elongate reservoir portion in each case.

5. The miniaturized inhalation device according to claim 1, wherein the first and the second elongate reservoir portion each have a cylindrical shape.

6. The miniaturized inhalation device according to claim 1, wherein the first and the second cavity each have a cylindrical shape.

7. The miniaturized inhalation device according to claim 1, wherein, in each clamping projection, a projection tip is rounded.

8. The miniaturized inhalation device according to claim 1, wherein each clamping projection has a triangular shape when the U-shaped component is viewed from the front.

9. The miniaturized inhalation device according to claim 1, wherein each clamping projection is formed such that it completely overlaps with the first and second elongate reservoir portions, respectively, in the longitudinal direction.

10. The miniaturized inhalation device according to claim 1, wherein each clamping projection has a smaller component thickness than the first and second elongate reservoir portions, when the U-shaped component is viewed from above.

11. The miniaturized inhalation device according to claim 1, wherein each clamping projection is arranged below and at a distance from a circumferential edge of the first and second top side openings, respectively.

12. The miniaturized inhalation device according to at least claim 1, wherein
the first cavity is provided with a first closure made of material permeable to the active ingredient, and
the second cavity is provided with a second closure made of material permeable to the active ingredient and/or the different active ingredient;
and/or
a plug is arranged in the first and/or the second cavity, in which the active ingredient and/or the different active ingredient are received such that they can be dispensed.

13. The miniaturized inhalation device according to claim 1, wherein the self-holding mechanism has an edge thickening on the outer side along the circumferential edge of the first and second top side openings, respectively.

14. The miniaturized inhalation device according to claim 1, wherein the first and second top side openings have an opening cross section which is substantially equal to a cavity cross section in the region of the first and the second cavity.

15. A set, comprising at least one miniaturized inhalation device according to claim 1 and a storage container for the at least one inhalation device, wherein the storage container is configured to accommodate the inhalation device, the storage container comprising a first receptacle and a second receptacle which are dimensioned and shaped to at least partly accommodate the first elongate reservoir portion and the second elongate reservoir portion therein, such that the first and second cavity are sealed against the environment.
